(19) United States
(12) Patent Application Publication
(10) Pub. No.: US 2011/0082386 A1
(43) Pub. Date: Apr. 7, 2011

(54) REPLACEMENT ADHESIVE UNIT FOR EAR COUPLERS

(76) Inventor: DAVID A. SHERATON, SR., San Clemente, CA (US)

(21) Appl. No.: 12/573,740

(22) Filed: Oct. 5, 2009

(51) Int. Cl. A61B 5/12; B32B 7/10; B32B 38/10

(52) U.S. Cl. 600/559; 428/354; 428/41.8; 156/247

(57) ABSTRACT

A replacement adhesive unit for optimizing adhesion to a disposable ear coupler and an ear of an infant comprises at least one adhesive layer that can be attached to a skin side flange of the disposable ear coupler, at least one structural tie layer to attach with a bottom side of the adhesive layer, and at least one adhesive ring having a top side and a bottom side, the top side being attached at a bottom surface of the structural tie layer. The structural tie layer provides structural support to the replacement adhesive unit and the application of at least one surface tape and the replacement adhesive unit in a laminated arrangement makes the disposable ear coupler reusable.

### BACKGROUND OF THE INVENTION

1. Technical Field of the Invention

The present invention relates in general to an adhesive unit of an ear coupler. More specifically, the present invention relates to a pressure sensitive replacement adhesive unit that makes an ear coupler reusable.

2. Description of the Related Art

A hearing test for infants is inevitable for assessing whether the infant is at a risk of having a hearing deficit. Various devices with sensors have been developed to test the hearing ability of an infant. It is beneficial to determine the existence of any hearing impairment at an early age so that appropriate treatments can be started. This minimizes the possible problems associated with the identified hearing impairments. Usually a hearing impairment is identified from the response signals generated by an earpiece with sensors in response to an input sound signal injected into the infant's ear. Ear couplers are used to attach these testing devices to the ear and are capable of being used with any conventional hearing screening equipment.

Traditional ear couplers available are generally disposable. Such a disposable ear coupler includes an annular side wall and a bottom wall forming an acoustic chamber. A flexible adhesive-backed flange is disposed on the periphery of the ear coupler that attaches to the infant's head holding the ear coupler in place over the ear. The annular side wall has a port for the placement of a transducer assembly, and also has ribs to help lock the transducer assembly in place. A common problem with these types of ear couplers is that the ear coupler is cost-ineffective in that the entire ear phone is not re-usable and must be disposed of after each use.

Another known type of ear coupler includes toroidal baffle of anechoic, insulative foam, a clear planar window plastic sheet adhered to one side of the baffle defining a cavity, and an adhesive coating such as hydrogel on the other side of the baffle. The ear coupler is capable of being used with transducers for transferring sound signal through a pneumatic tube to the infant's ear for testing. This type of ear coupler detaches from the infant's ear because of lose of adhesiveness of the coating after first use.

Hence, it can be seen, that there is a need for a replacement adhesive unit having a pressure sensitive adhesive layer for optimizing adhesion between an ear coupler and an ear of an infant. Further, the needed arrangement would make the disposable ear coupler reusable. Moreover, the needed replacement adhesive unit would be simple, economical and easy to use.

### SUMMARY OF THE INVENTION

To minimize the limitations found in the prior art, and to minimize other limitations that will be apparent upon the reading of the specifications, the present invention provides a replacement adhesive unit for at least one disposable ear coupler for optimizing adhesion to at least one disposable ear coupler and at least one ear of an infant. The unit comprises at least one adhesive layer having a top side and a bottom side to attach with a skin side flange of at least one disposable ear coupler, at least one structural tie layer to attach with the bottom side of the adhesive layer, and at least one adhesive ring having a top side and a bottom side, the top side being attached at a bottom surface of the structural tie layer. The structural tie layer provides structural support to the replacement adhesive unit and the application of at least one surface tape and the replacement adhesive unit in a laminated arrangement makes the disposable ear coupler reusable.

In another aspect of the present invention, a method in accordance with the present invention is a method of making at least one disposable ear coupler reusable and testing hearing problems of at least one infant comprising of providing at least one disposable ear coupler with at least one hydrogel ring, removing the hydrogel ring from the disposable ear coupler, applying at least one surface tape to a skin side flange of the disposable ear coupler to make the disposable ear coupler reusable, providing a replacement adhesive unit, peeling off at least one liner from the top side of at least one adhesive layer, adhering the top side of the adhesive layer to at least one reusable ear coupler, enclosing at least one ear of an infant by placing a cavity of the reusable ear coupler to the ear of an infant, coupling an outer surface of the cavity of the reusable ear coupler to at least one test equipment, screening hearing problems of the infant using the test equipment, removing the replacement adhesive unit from the reusable ear coupler, and disposing the replacement adhesive unit. After disposing the used replacement adhesive unit, a new replacement adhesive unit may be applied to a skin side flange of the reusable ear coupler for testing hearing problems of another infant.

One objective of the invention is to provide a replacement adhesive unit for making at least one disposable ear coupler reusable.

Another objective of the invention is to provide a replacement adhesive unit having a plurality of layers to form a laminated arrangement for optimizing adhesion to at least one disposable ear coupler and at least one ear of an infant.

A third objective of the invention is to provide a replacement adhesive unit having at least one adhesive layer on a first side and at least one adhesive ring that can be attached to a skin side flange of at least one disposable ear coupler and to at least one ear of an infant respectively.

A fourth objective of the invention is to provide a replacement adhesive unit having at least one structural tie layer to provide structural support to the replacement adhesive unit.

These and other advantages and features of the present invention are described with specificity so as to make the present invention understandable to one of ordinary skill in the art.

### BRIEF DESCRIPTION OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention, thus the drawings are generalized in form in the interest of clarity and conciseness.

FIG. 1 is an exploded perspective view of the preferred embodiment in accordance with the present invention;

FIG. 2 is an exploded perspective view of the another embodiment;

FIG. 3 is a pictorial flow chart of the preferred embodiment in accordance with the present invention showing a method of making at least one disposable ear coupler reusable; and

FIG. 4 is an operational flow chart of the preferred embodiment in accordance with the present invention illustrating a method of making at least one disposable ear coupler reusable and testing hearing problems of at least one infant.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

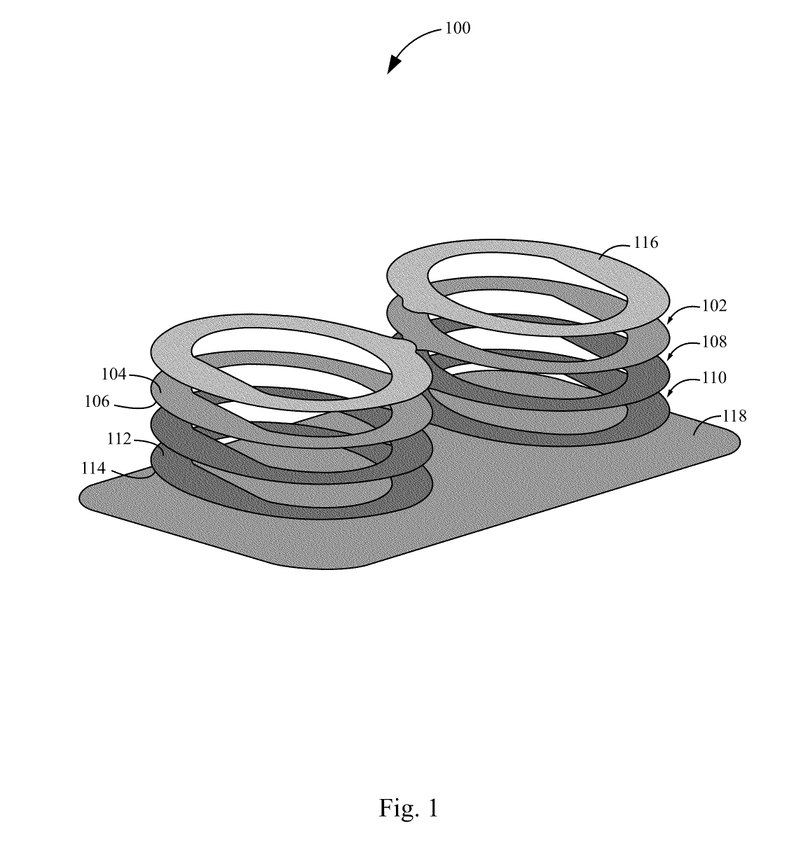

FIG. 1 is an exploded perspective view of the preferred embodiment in accordance with the present invention showing a replacement adhesive unit 100 attachable to at least one disposable ear coupler (not shown) for optimizing adhesion to the disposable ear coupler (not shown) and at least one ear of an infant (not shown). The replacement adhesive unit 100 comprises at least one adhesive layer 102 having a top side 104 to secure the replacement adhesive unit 100 to a skin side flange of the disposable ear coupler (not shown), at least one structural tie layer 108 to attach with a bottom side of at least one adhesive layer 106 and at least one adhesive ring 110 having a top side 112 and a bottom side 114. The bottom side of at least one adhesive ring 114 adheres to the replacement adhesive unit 100 to at least one ear of an infant (not shown) and the adhesive ring 110 may be a skin compatible two sided pressure sensitive adhesive, preferably a hydrogel. The replacement adhesive unit 100 further includes at least one liner 116 attached to a top side of the adhesive layer 104 and a liner card 118 laid below the bottom side of the adhesive ring 114. The liner 116 is used to protect the replacement unit 100 and the liner card 118 may be used to store the unit. The liner 116 attached to the top side of the adhesive layer 104 may be a release paper. The structural tie layer 108 provides structural support to the replacement adhesive unit100.

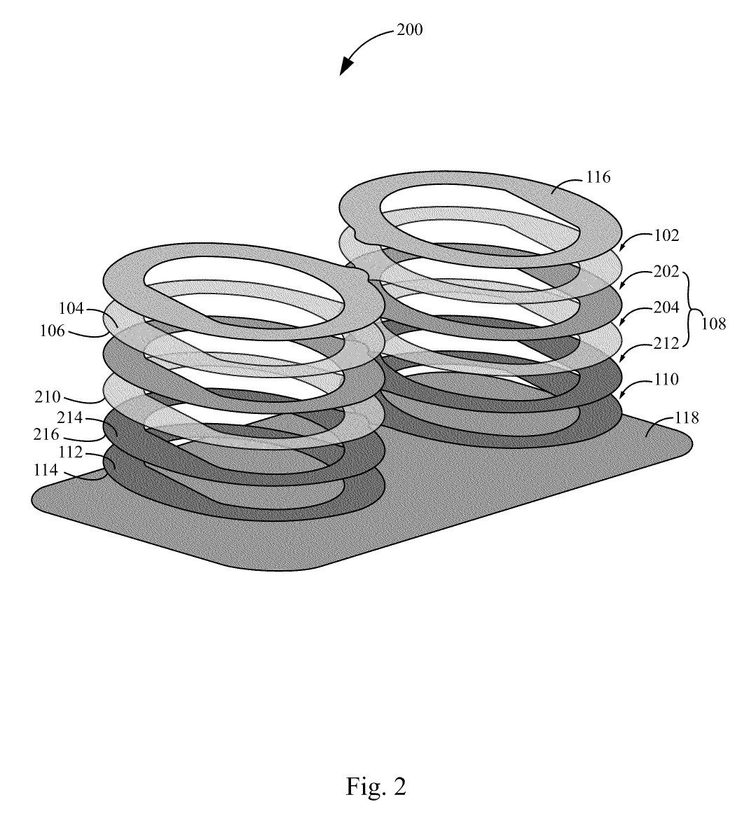

FIG. 2 is an exploded perspective view of an alternate embodiment in accordance with the present invention showing a replacement adhesive unit 200 attachable to at least one disposable ear coupler (not shown) for optimizing adhesion to the disposable ear coupler (not shown) and at least one ear of an infant (not shown). The replacement adhesive unit 200 comprises at least one adhesive layer 102 having a top side 104 to secure the replacement adhesive unit 200 to a skin side flange of the disposable ear coupler (not shown), at least one pressure sensitive adhesive 202, and at least one foam 204, at least one mesh 212 and at least one adhesive ring 110 having a top side 112 and a bottom side 114. In this embodiment the mesh 212, the pressure sensitive adhesive 202, and the foam 204 are arranged to form at least one structural tie layer 108. The foam 204 supports a bottom side of the pressure sensitive adhesive 202. The mesh 212 having a top surface 214 is affixed to the bottom side of the foam 210. The top side of the adhesive ring 112 attached to a bottom surface of the mesh 216 may be a polyester non-woven fabric and of thickness greater than the adhesive layer 102.

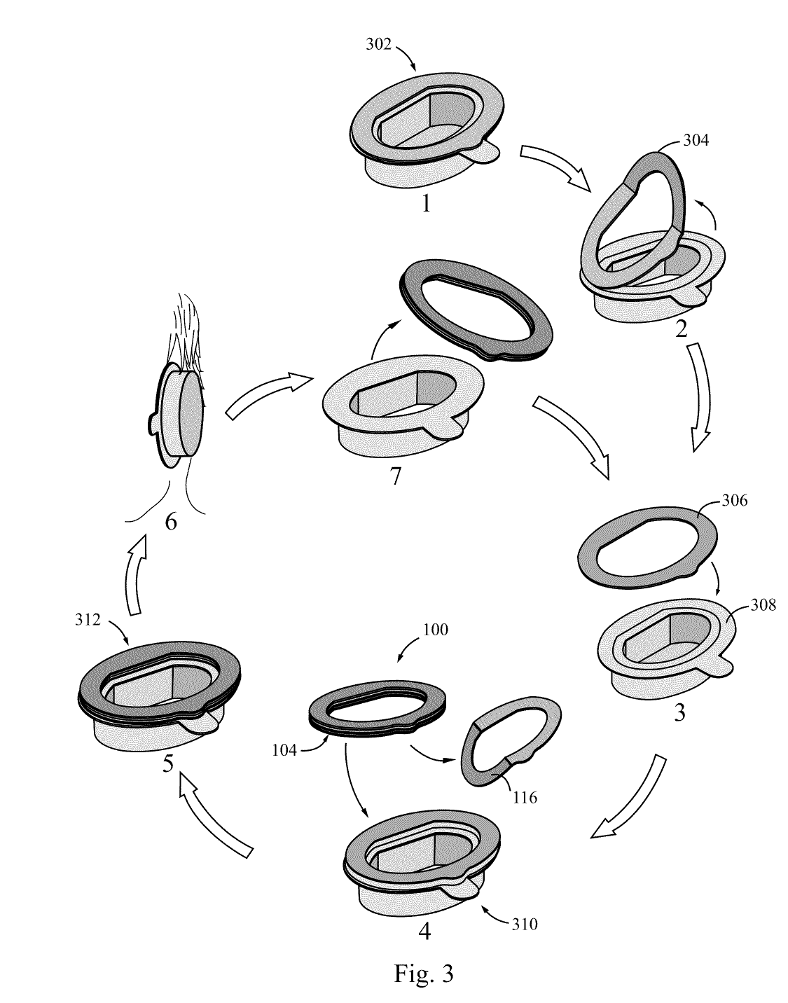

FIG. 3 is a pictorial flow chart of the preferred embodiment showing a sequence of steps for the method of making at least one disposable ear coupler 302 reusable. In the step 1, the disposable ear coupler 302 with at least one hydrogel ring 304 is provided. The hydrogel ring 304 is removed and at least one polyethylene (PE) tape 306 is applied to a skin side flange of the disposable ear coupler 308 making the disposable ear coupler 302 reusable as in steps 2 and 3. The PE tape 306 may be a single coated pressure sensitive adhesive. In step 4, the paper liner 116 is peeled off from the top side of the adhesive layer 104 and adhering the top side of the adhesive layer 104 to at least one reusable ear coupler 310. In step 5, the reusable ear coupler 312 is facilitated to apply on at least one ear of an infant. Next, as in step 6, the reusable ear coupler 312 is applied to the ear of an infant. After being applied to the ear of an infant, the replacement adhesive unit 100 may be disposed as depicted in step 7 and a new replacement adhesive unit may be applied to the reusable ear coupler 310.

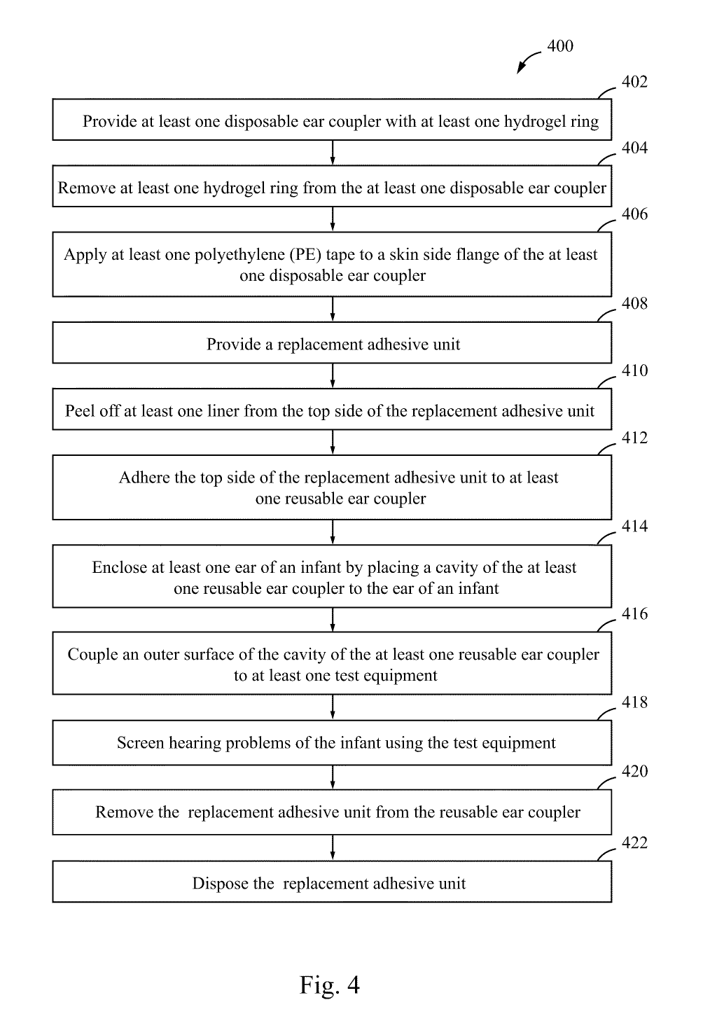

FIG. 4 shows an operational flow chart 400 of the preferred embodiment in accordance with the present invention illustrating a method of making at least one disposable ear coupler reusable and testing hearing problems of at least one infant. The disposable ear coupler with at least one hydrogel ring is provided as indicated at block 402. The hydrogel ring is removed as indicated at block 404. At least one polyethylene (PE) tape is applied to a skin side flange of the ear coupler to make the ear coupler reusable as indicated at block 406. A replacement adhesive unit is provided as indicated at block 408. Then, peel off the liner from the top side of the adhesive layer as indicated at block 410. The top side of the adhesive layer is adhered to at least one reusable ear coupler as indicated at block 412. The ear of an infant is enclosed by placing a cavity of the reusable ear coupler to the ear of an infant as indicated at block 414. An outer surface of the cavity of the reusable ear coupler is coupled to at least one test equipment as indicated at block 416. The hearing problems of the infant are screened using the test equipment as indicated at block 418. The replacement adhesive unit after being applied to the ear of an infant is removed from the reusable ear coupler as depicted at block 420 and finally, the replacement adhesive unit can be disposed as indicated at block 422.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention not be limited by this detailed description, but by the claims and the equivalents to the claims appended hereto.

What is claimed is:

1. A replacement adhesive unit for at least one disposable ear coupler for optimizing adhesion to at least one disposable ear coupler and at least one ear of an infant, the replacement adhesive unit comprising:
at least one adhesive layer having a top side and a bottom side, at least one adhesive layer secures the replacement adhesive unit to a skin side flange of at least one disposable ear coupler;
at least one structural tie layer to attach with the bottom side of at least one adhesive layer; and
at least one adhesive ring having a top side and a bottom side, the top side being attached at a bottom surface of at least one structural tie layer;
whereby at least one structural tie layer provides structural support to the replacement adhesive unit.

2. The replacement adhesive unit of claim 1 wherein the structural tie layer can be a single layer or multilayered.

3. The replacement adhesive unit of claim 2 wherein the multilayers can be at least one mesh, at least one pressure sensitive adhesive and at least one foam.

4. The replacement adhesive unit of claim 3 wherein at least one mesh can be a polyester non-woven fabric.

5. The replacement adhesive unit of claim 1 wherein the top side of at least one adhesive layer is attached to at least one liner to protect the replacement adhesive unit.

6. The replacement adhesive unit of claim 5 wherein at least one liner can be a release paper.

7. The replacement adhesive unit of claim 1 wherein at least one adhesive ring can be a skin compatible, pressure sensitive adhesive (PSA).

8. The replacement adhesive unit of claim 1 wherein at least one adhesive ring can be a two sided pressure sensitive adhesive coated foam tape.

9. The replacement adhesive unit of claim 1 wherein at least one adhesive ring can be a hydrogel.

10. The replacement adhesive unit of claim 1 wherein at least one adhesive ring can be of thickness lesser than at least one mesh.

11. The replacement adhesive unit of claim 1 wherein the bottom side of at least one adhesive ring adheres the replacement adhesive unit to at least one ear of an infant.

12. The replacement adhesive unit of claim 1 wherein the skin side flange of the one disposable ear coupler is applied with at least one surface tape that can be a single coated pressure sensitive adhesive.

13. The replacement adhesive unit of claim 14 wherein the application of at least one surface tape makes the disposable ear coupler reusable.

14. The replacement adhesive unit of claim 1 wherein the replacement adhesive unit is laid in at least one liner card until use.

15. The replacement adhesive unit of claim 1 wherein the replacement adhesive unit is disposable.

16. A method of making at least one disposable ear coupler reusable and testing hearing problems of at least one infant, the method comprising the steps of:
(a) providing at least one disposable ear coupler with at least one hydrogel ring;
(b) removing the hydrogel ring from at least one disposable ear coupler;
(c) applying at least one surface tape to a skin side flange of the disposable ear coupler to make the disposable ear coupler reusable;
(d) providing a replacement adhesive unit;
(e) peeling off at least one liner from top side of at least one adhesive layer;
(f) adhering the top side of at least one adhesive layer to at least one reusable ear coupler;
(g) enclosing at least one ear of an infant by placing a cavity of the reusable ear coupler to the ear of an infant;
(h) coupling an outer surface of the cavity of the reusable ear coupler to at least one test equipment;
(i) screening hearing problems of at least one infant using at least one test equipment;
(j) removing at least one replacement adhesive unit from the reusable ear coupler; and
(k) disposing the replacement adhesive unit.

17. The method of claim 16 wherein the steps (a) through (k) may be repeated.
